(19) United States
(12) Patent Application Publication
(10) Pub. No.: US 2023/0138457 A1
(43) Pub. Date: May 4, 2023

(54) ANATOMIC SIMULATION ASSEMBLIES

(71) Applicant: INTUITIVE SURGICAL OPERATIONS, INC., Sunnyvale, CA (US)

(72) Inventors: Matthew D. Rohr Daniel, San Francisco, CA (US); Ruchi C. Bhatt, San Jose, CA (US); Leah Muller, San Francisco, CA (US); Thomas Roberts, Campbell, CA (US); Alexis Veracruz, Medford, MA (US)

(21) Appl. No.: 17/976,636

(22) Filed: Oct. 28, 2022

Related U.S. Application Data
(60) Provisional application No. 63/273,198, filed on Oct. 29, 2021.

(51) Int. Cl. G09B 23/30; G16H 50/50

(52) U.S. Cl. CPC G09B 23/30; G16H 50/50

(57) ABSTRACT

Anatomic simulators, assemblies, and related methods are disclosed. In some embodiments, an anatomic simulator may include constraint panels that define a variably sized volume configured to receive an organ model. An elastic member may be configured to provide a restorative force when the constraint panels are moved to an expanded configuration. Alignment apertures in the constraint panels may be configured to permit marking of an organ model and/or visibility of a mark disposed on the organ model to position and orient the organ model in the anatomic simulator. In some embodiments, an anatomic simulation assembly may include an anatomic simulator, a pressure source, pneumatic controls, a pressure sensor, and a processor. In some embodiments, a method of simulating anatomic motion may include flowing a fluid to an organ model, sensing a pressure, and controlling the flow of fluid based on the sensed pressure to maintain a target pressure.

### CROSS-REFERENCED APPLICATIONS

This application claims priority to and benefit of U.S. Provisional Application No. 63/273,198, filed Oct. 29, 2021 and entitled “Anatomic Simulation Assemblies,” which is incorporated by reference herein in its entirety.

### FIELD

Disclosed embodiments are related to anatomic simulators and anatomic simulation assemblies.

### BACKGROUND

Anatomic simulators and anatomic simulation assemblies give medical professionals (operators, surgeons, physicians, technicians, medical assistants, etc.) the ability to practice a procedure in a low-risk environment before performing the procedure on an actual patient. To more closely represent the conditions of an actual medical operation, some anatomic simulators attempt to mimic certain anatomic structures and/or motions.

### SUMMARY

In some embodiments, an anatomic simulator comprises a first constraint panel configured to rotate about a first axis and a second constraint panel configured to rotate about a second axis. The first and second constraint panels at least partially define a variably sized volume configured to receive an organ model. The anatomic simulator further comprises at least one elastic member operatively coupling the first and second constraint panels. The at least one elastic member is configured to provide a restorative force when the first and second constraint panels are rotated to increase a size of the volume.

In some embodiments, an anatomic simulator comprises one or more constraint panels that at least partially define a volume configured to receive an organ model and at least one alignment aperture formed in the one or more constraint panels. The at least one alignment aperture is configured to permit marking of the organ model when the organ model is received within the anatomic simulator and/or visibility of at least one mark disposed on the organ model when the organ model is received within the anatomic simulator to provide a desired position and orientation of the organ model relative to the one or more constraint panels when the organ model is received within the anatomic simulator.

In some embodiments, an anatomic simulation assembly comprises an anatomic simulator configured to receive an organ model and a pressure source in fluid communication with the anatomic simulator. The pressure source is configured to flow a fluid from the pressure source to the organ model when the organ model is received within the anatomic simulator. The anatomic simulation assembly further comprises one or more pneumatic controls disposed along a flow path between the pressure source and the organ model when the organ model is received within the anatomic simulator, a pressure sensor configured to sense a pressure of the fluid provided to the organ model when the organ model is received within the anatomic simulator, and a processor operatively coupled to the one or more pneumatic controls and the pressure sensor. The processor is configured to operate in a first mode of operation in which the processor controls the flow of fluid from the pressure source to the organ model with the one or more pneumatic controls based at least partially on the sensed pressure to maintain a target pressure within the organ model.

In some embodiments, a method of simulating anatomic motion comprises flowing a fluid from a pressure source to an organ model within an anatomic simulator, sensing a pressure of the fluid provided to the organ model, and controlling the flow of fluid to the organ model based at least partially on the sensed pressure to maintain a target pressure within the organ model.

In some embodiments, a system comprises a simulator including an interior volume configured to receive an organ model, an instrument including a sensor system for capturing localization data, a structure configured for insertion within the interior volume, a processor operably coupled to the sensor system, and a memory operably coupled to the processor. The memory stores instructions that, when executed by the processor, cause the system to perform operations comprising receiving imaging data of the simulator and the organ model, generating a 3D virtual model of the organ model, and receiving the localization data while the instrument is positioned within the organ model.

In some embodiments, a method comprises receiving an organ model within an interior volume of a simulator, receiving imaging data of the simulator and the organ model, generating a 3D virtual model of the organ model, inserting a structure within the interior volume of the organ model, and receiving localization data captured by a sensor system of an instrument while the instrument is positioned within the organ model.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a perspective view of one embodiment of an anatomic simulator.

FIG. 2 is a top view of the anatomic simulator of FIG. 1.

FIG. 3 is a side view of the anatomic simulator of FIG. 1.

FIG. 4 is a side view of a constraint panel of the anatomic simulator of FIG. 1.

FIG. 5A depicts an organ model disposed in one embodiment of an anatomic simulator in an open configuration.

FIG. 5B depicts the anatomic simulator of FIG. 5A in a closed configuration.

FIG. 6A depicts a lung model and a heart structure.

FIG. 6B depicts the lung model and heart structure of FIG. 6A disposed within one embodiment of an anatomic simulator.

FIG. 7 is a schematic of one embodiment of an anatomic simulation assembly.

FIG. 8 is a flowchart of one embodiment of a method of simulating anatomic motion.

FIG. 9 is a perspective view of an example of an anatomic simulator including a simulation insert member.

FIGS. 10A and 10B are perspective views of an example of an anatomic simulator including simulation insert members and a laryngeal mask airway (LMA).

### DETAILED DESCRIPTION

Before performing a procedure on a human patient, medical professionals may train using models. Live animal and/or cadaveric models may be used for training, as these models may accurately replicate the anatomy that would be encountered during an actual procedure. However, the process of obtaining animal and/or cadaveric models is often time intensive, logistically challenging, and expensive.

As an alternative to live animal and/or cadaveric models, anatomic simulations may be used. These simulations may recreate the conditions of an actual procedure, and yet may avoid the difficulty of obtaining an animal and/or cadaveric model. Instead of using a live animal and/or cadaver, an anatomic simulation may include only a single organ that is received within a training system (i.e., the anatomic simulator) that replicates the static and dynamic anatomical conditions that may be encountered by the physician during an actual case. That is, an anatomic simulator may be an engineered medical system that is configured to receive an animal or cadaveric organ and interact with the organ to express anatomical motions to the physician, thereby resulting in a system that simulates an organ and its surrounding anatomy in a living patient.

Despite the many advantages related to reduced cost and increased convenience, conventional anatomic simulators are often associated with certain limitations. For example, conventional anatomic simulators often fail to replicate true anatomic motion. During an actual operation, medical professionals interact with organs that move dynamically as they perform their natural, biological functions. For example, lungs expand as the patient inhales (or as a ventilator forces air into the lungs), and the heart contracts as it pumps blood throughout the patient's body. While some conventional anatomic simulators may enable the organ model received within the simulator to expand and/or contract to some degree, the physical structure of the simulator itself may restrict or otherwise alter the natural motion of the organ. Alternatively, some conventional anatomic simulators may enable the organ model received within the simulator to expand and/or contract freely without any external constraints. Neither simulator (i.e., a simulator with rigid constraining structures or a simulator without any constraining structures) may accurately reproduce the organ's natural motion. For example, a person's rib cage is sufficiently compliant to dynamically accommodate the expansion and contraction of the lungs during respiration. However, conventional anatomic simulators configured to simulate respiration may not include compliant structures that replicate the natural compliance of the rib cage. Instead, conventional anatomic simulators often include rigid constraining structures, or do not include any constraining structures at all. With rigid physical constraints, the organ may not exhibit the full range of natural anatomic motion while it is contained within the simulator. In contrast, without any physical constraints, the organ may exhibit motion that exceeds the range of natural anatomic motion while it is contained within the simulator. As such, a physician training on the simulator may not be provided with an experience that is representative of an actual procedure.

In view of the above, the inventors have recognized and appreciated the benefits associated with an anatomic simulator that enables true anatomic motion. The inventors have recognized that an anatomic simulator that includes structures that move in sync with the organ model as the organ model expands or contracts may more realistically approximate natural anatomic motion. Compared to conventional anatomic simulators that may include rigid and/or static structures, some embodiments of anatomic simulators described herein may include dynamic structures that translate, rotate, bend, and/or flex to accommodate the natural motion of the organ model and more accurately mimic the inherent compliance of the actual anatomic structures that would surround the organ in a live patient.

In order to provide the above desired functionality, in some embodiments, an anatomic simulator may include one or more panels configured to constrain motion of an organ model when the organ model is disposed within the anatomic simulator. The constraint panels may be configured to replicate anatomic features. In the case of an anatomic simulator configured to receive a lung model, for example, separate left and right constraint panels may simulate a rib cage. As such, the constraint panels in such an embodiment may be structured and arranged to promote primary filling of a lung model in a direction toward the lower lobes of the lung model received therein while partially constraining expansion of the lungs in one or more other directions. Each constraint panel may be coupled to a base of the anatomic simulator, such that each constraint panel may be moveable relative to the base via an appropriate flexible and/or rotatable connection. As such, in some instances, the constraint panels may be arranged in a clamshell configuration where at least a portion of the constraint panels are configured to rotate about two corresponding rotation axes which need not be collinear, parallel, or aligned. In some embodiments, an anatomic simulator may include a first constraint panel configured to rotate about a first axis and a second constraint panel configured to rotate about a second axis.

The constraint panels may at least partially define a variably sized volume configured to receive an organ model. When an organ model disposed within the volume is expanded from a retracted configuration to an expanded configuration, the expanding organ model may exert a force on each of the constraint panels, thereby causing the constraint panels to rotate about their axes and expand the variably sized volume from a first volume to a second larger volume, thereby providing additional space for the expanding organ model and more accurately reflecting true anatomic motion of a complaint rib cage.

In some embodiments, an anatomic simulator may also include at least one elastic member that is configured to store potential energy when the constraint panels move from a first configuration to a second configuration. For example, when the organ model expands and causes the constraint panels to rotate away from their initial configuration, the at least one elastic member may store elastic potential energy. When the organ model retracts to its retracted configuration, the at least one elastic member may use its stored elastic potential energy to provide a restorative force that causes the constraint panels to return to their initial configuration.

In some embodiments, an anatomic simulator may enclose an organ model within a sealed environment. A sealed environment may enable temperature and/or humidity control, thereby better representing the conditions of an actual medical procedure. In addition to being more realistic, an anatomic simulator with temperature and/or humidity control may better preserve the natural functions and/or structural characteristics of an organ model. For example, a warm moist lung model may expand more naturally and to its regular extent in response to an applied pressure compared with a dry and/or cold lung model.

While some conventional anatomic simulators may be able to accommodate some amount of motion of the organ model, such simulators are typically only able to replicate a single motion pattern. For example, an anatomic simulator configured to replicate respiration using a lung model may only be configured to replicate regular, steady respiration. However, in actual medical procedures, a medical professional often desires more control over the breathing pattern of the patient. For example, the medical professional may adjust a ventilator to enter a “breath hold” mode, in which the lung (or other organ) is held in a static condition (e.g., at a constant pressure). Such a breath hold mode may be particularly advantageous when a delicate or sensitive task is being undertaken and a static working environment is desired. When the task is accomplished, the ventilator may be toggled back to a normal breathing mode, in which the lung is cyclically inflated and deflated. It may be beneficial to provide breath holds to simulate real animal lab or procedural situations. Performing breath holds may be challenging for conventional anatomic simulators. For example, if an organ model includes defects (e.g., small perforations), the anatomic simulator may be unable to maintain a constant pressure and perform a breath hold due to undetected leaks in the organ model and insufficient air flow control to compensate for these defects.

In view of the above, the inventors have recognized and appreciated the benefits associated with an anatomic simulation assembly that includes pressure sensing and feedback control to enable different breathing modes, including but not limited to breath holds. In some embodiments, such an assembly may include an anatomic simulator configured to receive an organ model, a pressure source in fluid communication with the anatomic simulator and configured to flow a fluid to the organ model, and pneumatic controls disposed along a flow path between the pressure source and the organ model. A pressure sensor may be configured to sense a pressure of the fluid provided to the organ model within the anatomic simulator, and a processor coupled to the pneumatic controls and the pressure sensor may be configured to operate the pressure source and/or pneumatic controls in various modes. In a breath hold mode, the processor may be configured to control the flow of fluid from the pressure source to the organ model with the one or more pneumatic controls based at least partially on the sensed pressure to maintain a target pressure within the organ model. In a normal breathing mode, the processor may be configured to control the flow of fluid from the pressure source to the organ model with the one or more pneumatic controls based at least partially on the sensed pressure and one or more parameters to cyclically increase and decrease a pressure within the organ model. Such an anatomic simulation assembly may be able to provide the desired functionality while accommodating for unknown numbers and/or types of defects in the organ model.

Furthermore, it can be desirable in a training exercise to accurately repeat a full image guided medical procedure performed on a patient using an organ model received within a simulator. In an image guided medical procedure, patient anatomy may be pre-operatively imaged (using, for example, a computerized tomography (CT) scan, a magnetic resonance imaging (MRI) scan, or other suitable imaging technique), and a 3D virtual model of the anatomy may be generated. Using this 3D virtual model, certain aspects of the procedure may be visualized and planned. For example, the 3D virtual model may be used to select a target, or plan a path to a target. Intra-operatively, the medical professional may navigate medical instruments with localization sensors within the patient anatomy. By capturing in real time position data from the instrument's localization sensors, the medical instruments may be registered to the 3D virtual model and real-time guidance along the pre-planned path to the target may then be provided to assist the medical professional in navigating the instrument within the patient. Similarly, during a training exercise, in order to replicate an image guided medical procedure using an organ model, the organ model can be imaged within the simulator to generate a 3D virtual model of the organ model to be used in to create a pre-planned path to a target and provide real time guidance during a training procedure performed within the organ model. Often, an organ model is moved between different simulators at different locations, or removed and replaced within the same simulator at different times. It is desirable to use the same 3D virtual model and planned path for the same organ model when moved from or replaced to a simulator or moved to a different simulator, eliminating the need to capture new image data and create a new 3D model and planned path. Additionally, imaging during a real patient procedure may occur days or weeks before the actual medical procedure, so it is reasonable to assume that organ position, orientation, and/or pose relative to the simulator may change. Accordingly, it can be desirable to simulate a predicted shift in patient anatomy between when imaging data is captured and a performed medical procedure. Conventional simulators are limited in that they do not facilitate accurate and repeatable alignment between the organ model and the simulator and often fail to provide any indication of a shifted alignment between the organ and the simulator from an imaging stage to a procedure stage.

In view of the above, the inventors have recognized and appreciated the benefits associated with an anatomic simulator that enables repeatable and reliable image guidance within an organ model. The inventors have appreciated that it would be advantageous to be able to position an organ model within an anatomic simulator during a medical training procedure to be consistent with the pose of the organ model within the anatomic simulator during the pre-operative imaging procedure and in some cases, shift the pose of the organ model to accommodate predicted shifts that could occur during real patient procedures. The medical professional may be confident that a procedure planned using a 3D virtual model (derived from the pre-operative imaging) would be appropriately applied to the physical organ model during the training procedure.

Accordingly, some embodiments of an anatomic simulator may include at least one alignment aperture formed in a constraint panel. Initially, the alignment aperture may permit marking of the organ model when the organ model is first received within the anatomic simulator, such as during an imaging procedure. Subsequently, the alignment aperture may permit visibility of the mark(s) disposed on the organ model when the organ model is received within the same (or a different) anatomic simulator. Visibility of the mark(s) may provide an indication of a desired position and orientation of the organ model relative to the one or more constraint panels when the organ model is received within the anatomic simulator.

As used herein, the term “position” should be understood as relating to a translational parameter, such as the linear coordinates of a point along a coordinate system's axes. The term “orientation” should be understood as relating to a rotational parameter, such as the angular coordinates of a body around a coordinate system's axes. The term “pose” should be understood as encompassing both the position and orientation of a body.

As used herein, rotation of a constraint panel about a connection to a corresponding base (e.g., an axis of rotation) may include both free rotation of the constraint panel about a connection that freely permits rotation of the constraint panel about the axis of rotation as well as elastic deformation of the constraint panel, the base, and/or an intermediate component that permits at least a portion of the constraint panel to rotate about the axis of rotation. Appropriate types of rotatable connections may include, but are not limited to, living hinges (e.g., elastic portions of the base, constraint panel, and/or an intermediate elastic component), torsional springs disposed between the constraint panel and base, mechanical hinges disposed between the constraint panel and base, and/or any other appropriate rotatable connection that is capable of connecting a constraint panel to a corresponding base while permitting at least a portion of the constraint panel to rotate relative to the base.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

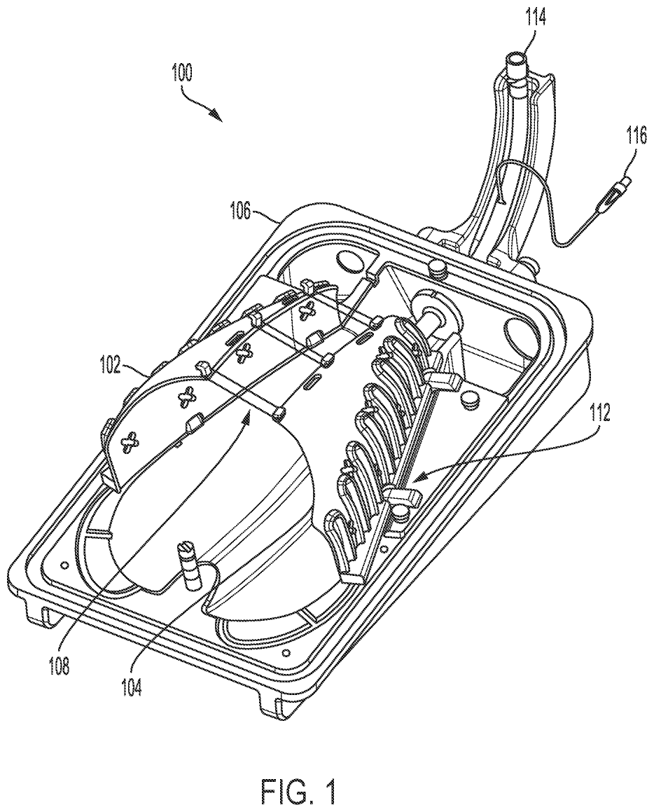

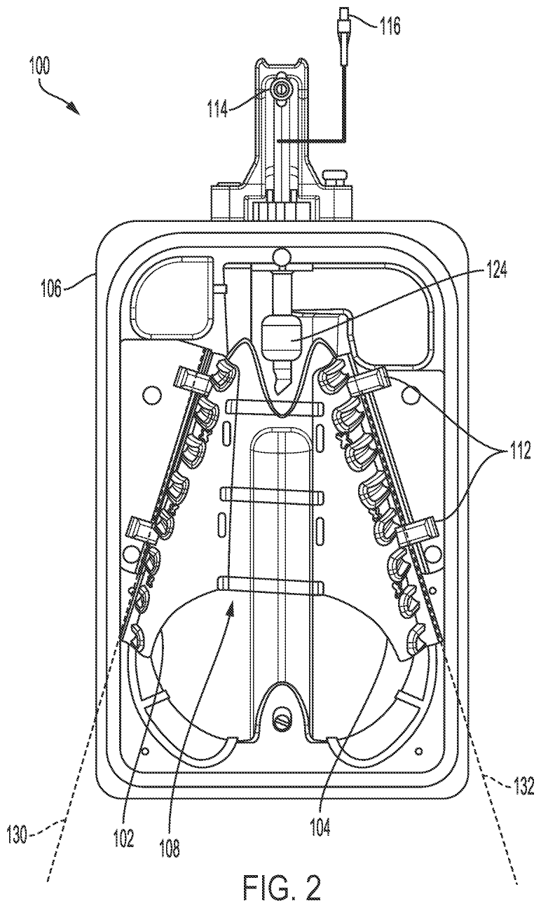

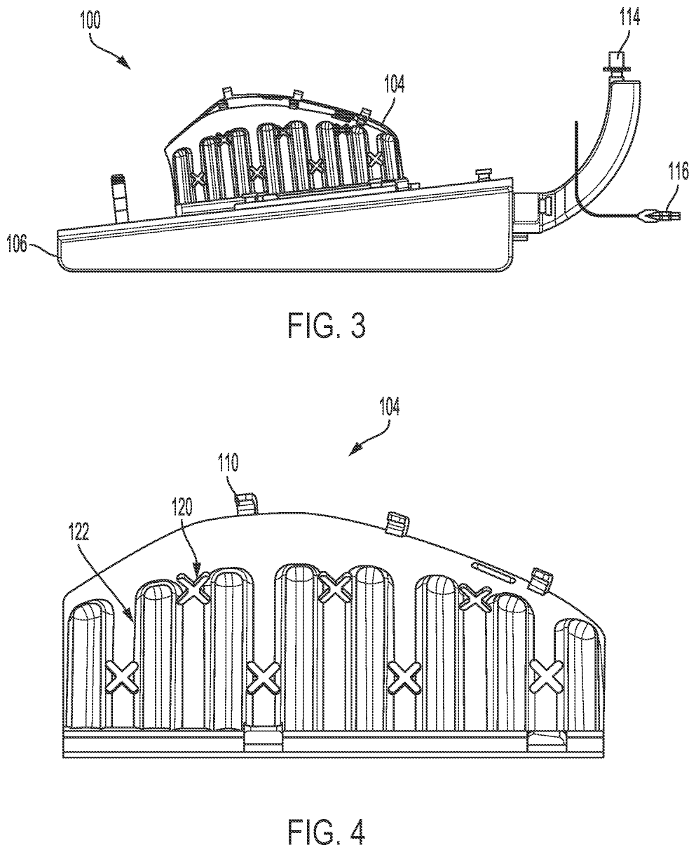

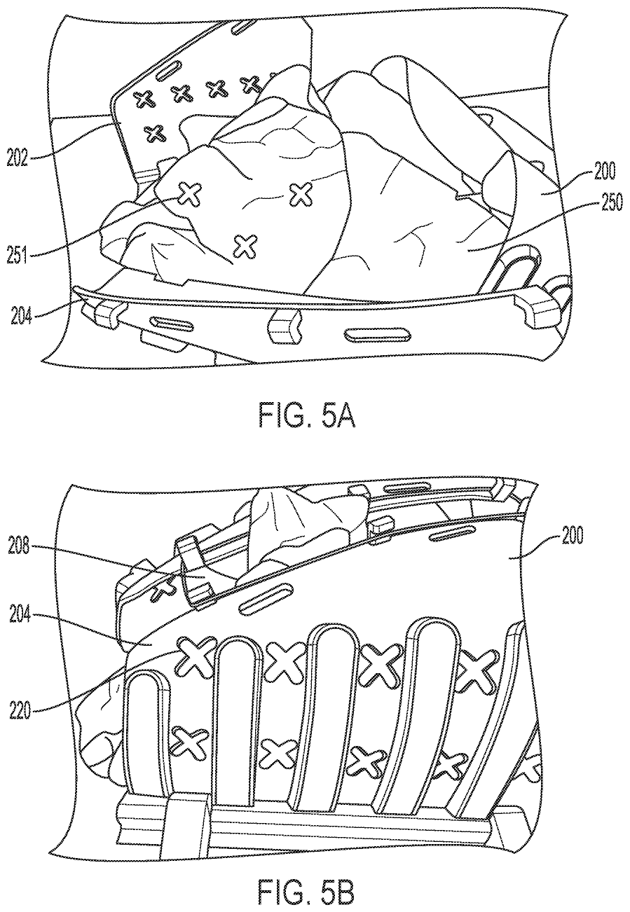

FIGS. 1-3 present different views of one embodiment of an anatomic simulator 100. The anatomic simulator 100 includes a first constraint panel 102 and a second constraint panel 104 (FIG. 4 is a side view of the second constraint panel 104 in isolation). The anatomic simulator 100 additionally includes a base 106 configured to support an organ model when the organ model is received within the anatomic simulator 100. Each constraint panel 102, 104 is rotatably coupled to the base 106, and is configured to rotate relative to the base 106 about a respective axis. For example, when a lung model received within the simulator, expansion of the lung model may cause the constraint panels 102 and 104 to rotate about their respective axes to provide additional space for the expanding lung model. As seen in FIG. 2, the first constraint panel 102 is configured to rotate about a first axis 130, and the second constraint panel 104 is configured to rotate about a second axis 132. When an organ model disposed within the anatomic simulator 100 is pressurized, the first constraint panel 102 rotates about the first axis 130 in a first direction and the second constraint panel 104 rotates about the second axis 132 in a second direction that is directed at least partially opposite from the first direction such that the first and second constraint panels are displaced towards an expanded configuration. For example, in some embodiments, as the first and second constraint panels rotate about their connections 112 to the base, the constraint panels may rotate away from each other. When the organ model is depressurized, the first constraint panel 102 rotates about the first axis 130 in a third direction opposite the first direction, and the second constraint panel 104 rotates about the second axis 132 in a fourth direction opposite the second direction such that the first and second constraint panels are displaced towards a retracted configuration. For example, in some embodiments, as the first and second constraint panels rotate about their connections 112 to the base, the constraint panels may rotate towards each other. In the embodiment of the figures, the first axis 130 is angled relative to and offset from the second axis 132. In other embodiments, the two axes may be parallel, aligned, collinear, angled, offset, skew, or otherwise arranged, as the present disclosure is not limited to the relative orientation of the two axes.

In some embodiments, the anatomic simulator 100 may additionally include at least one elastic member 108 configured to bias the first constraint panel 102 and the second constraint panel 104 towards the retracted configuration. Specifically, the elastic members 108 are configured to provide a restorative force when the first and second constraint panels 102, 104 are rotated to increase a size of the volume. In some embodiments, the size of the volume may be increased when the two constraint panels are rotated away from one another. The restorative force provided by the elastic members 108 biases the first constraint panel 102 and the second constraint panel 104 towards the noted retracted configuration. As best seen in FIG. 4, each constraint panel 102, 104 includes anchors 110 that may receive the elastic members 108. The anchors 110 may be hooks, posts, other suitable projections, and/or any other structure capable of retaining a corresponding elastic member 108. In the embodiment of the figures, three separate annular elastic members 108 couple adjacent opposing portions of the first constraint panel 102 and the second constraint panel 104 that are oriented at least partially towards one another. However, it should be appreciated that other numbers and/or arrangements of elastic members may be appropriate, and that the present disclosure is not limited in this regard. For example, rather than annular elastic members 108 directly coupling the first and second constraint panels 102, 104, an anatomic simulator may include: torsional springs, elastic living hinges, and/or other spring like connections between the constraint panels and the base; integrated elastic structures coupling the constraint panels to one another; and/or any other appropriate type of elastic member capable of providing a restorative torque when the constraint panels are rotated or otherwise displaced away from one another.

The anatomic simulator 100 includes a fluid inlet 114. The fluid inlet 114 is configured to be connected to and direct fluid (e.g., pressurized air) from a separate pressure source to an organ model disposed within the anatomic simulator 100. Appropriate types of pressure sources may include, but are not limited to, a volume of compressed fluid (e.g., a pressurized cylinder), a positive displacement pump, a bellows pump, and/or any other appropriate pressure source that may be connected to the fluid inlet. The anatomic simulator may also include a fluid outlet 124, see FIG. 2, that is in fluid communication with the fluid inlet. The fluid outlet may be appropriately sized and shaped to form a seal against an opening of the organ model when the organ model is positioned in the anatomic simulator. In embodiments where the organ model expands and contracts, such as a lung model, the lung model may be cyclically inflated and deflated to mimic inhalation and expiration. Specifically, a fluid such as air may be provided to the lung model via the fluid inlet 114 and corresponding fluid outlet 124 when the lung model is disposed within the anatomic simulator 100. In some embodiments, the anatomic simulator 100 includes a sensor 116. The sensor 116 may be configured to sense one or more parameters relating to the organ model when the organ model is disposed within the anatomic simulator. For example, the sensor 116 may be a pressure sensor that is operatively coupled to a portion of the flow path between the fluid inlet and outlet thereby enabling sensing of the pressure of the organ model. An indirect coupling between the sensor and fluid path extending between the fluid inlet and fluid outlet is shown by a sampling tube in the depicted embodiment. However, it should be understood that any appropriate type of sensor and arrangement of the sensor, including both indirect and direct sampling configurations, may be used as the disclosure is not limited in this manner.

Turning to FIG. 4, reference is made to the second constraint panel 104, but it should be appreciated that the first constraint panel 102 may be similarly arranged. The constraint panel 104 includes at least one alignment aperture 120 formed in the constraint panel. The alignment aperture 120 is configured to permit marking of an organ model when the organ model is received within the anatomic simulator 100 and/or visibility of a mark disposed on the organ model when the organ model is received within the anatomic simulator 100. Visibility of a mark on the organ model through the alignment aperture 120 may provide a desired position and orientation of the organ model relative to the constraint panel 104 when the organ model is received within the anatomic simulator 100. Although the embodiment of the figure depicts two offset rows of x-shaped alignment apertures 120, it should be appreciated that different shapes, numbers, and/or arrangements of alignment apertures may be appropriate, and that the present disclosure is not limited in this regard. For example, an alignment aperture may be a cross, an x-shape, a dot, a circle, a triangle, a square, a star, or any other suitable shape. Alignment apertures may be arranged in an array (e.g., a rectilinear array, a hexagonal array, an offset array, etc.), or in another regular and/or repeating pattern. In some embodiments, alignment apertures may be arranged in a non-repeating and/or irregular arrangements. Additionally, it should be appreciated that the shape, number, and/or arrangement of alignment apertures on the first constraint panel 102 may be the same or different than the shape, number, and/or arrangement of alignment apertures on the second constraint panel 104.

In some embodiments, a constraint panel may include one or more portions that are radiopaque. These radiopaque portions may be representative of anatomic features, which may enable a more realistic training environment. Additionally, in some embodiments, the radiopaque portions of a constraint panel may aid in registration of a 3D virtual model to a physical anatomic simulator. In the embodiment of FIG. 4, the constraint panel 104 includes multiple radiopaque portions 122 that are shaped and arranged to simulate the presence of the individual ribs of a ribcage.

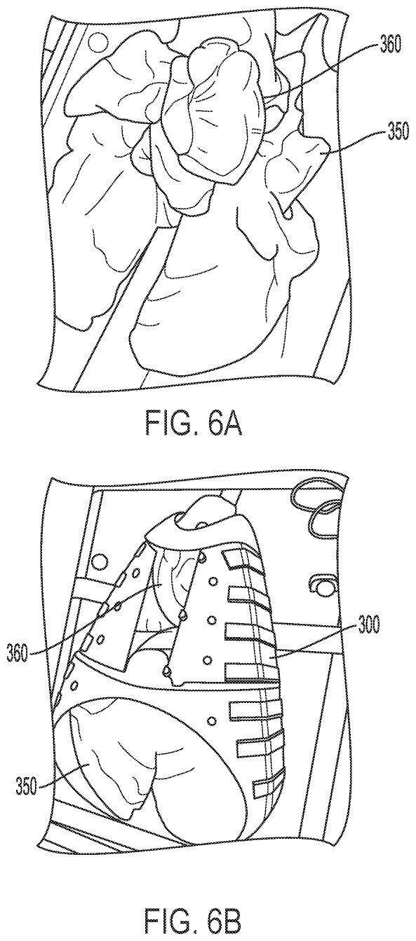

FIGS. 5A and 5B depict an organ model 250 disposed in one embodiment of an anatomic simulator 200. Specifically, a lung model 250 is disposed within the volume of the simulator 200 defined by a first constraint panel 202 and a second constraint panel 204. FIG. 5A shows the anatomic simulator 200 in an open configuration in which the two constraint panels 202, 204 are in an open configuration for receiving the lung model. In this open configuration, the elastic members have been removed, thereby removing the restorative force that would otherwise urge the constraint panels 202, 204 toward a retracted configuration. In the open configuration, a mark 251 on the lung model 250 is clearly visible. The mark 251 may be any permanent visual marking that does not impact the tissue of the lung model and that is compatible with materials used in storage and/or manipulation of the organ model, such as water and formaldehyde solutions. The mark may be a colored flexible polymer or a cyanoacrylate in some embodiments. FIG. 5B shows the anatomic simulator 200 in a closed configuration in which the two constraint panels 202, 204 are in a closed and retracted configuration with elastic members 208 connected to and biasing the constraint panels towards the retracted configuration. In the closed configuration, the mark 251 on the lung model 250 is visible through an alignment aperture 220 in the constraint panel 204, thereby providing indication of a predetermined alignment between the organ model 250 and the anatomic simulator 200.

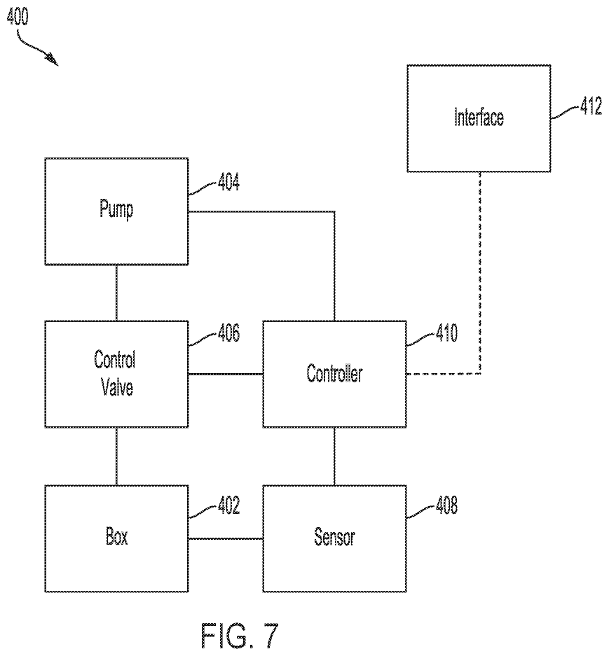

In some embodiments, an anatomic simulator may be configured to receive one or more additional structures within the interior volume of the anatomic simulator in addition to an organ model. Additional structures may be representative of anatomical structures present in a normal functioning human, and may aid in making a simulation more representative of an actual medical procedure by better approximating actual anatomy. For example, FIG. 6A depicts a lung model 350 and a heart structure 360. The heart structure 360 may have a size, shape, and/or stiffness that is representative of an actual heart. When the lung model 350 and the heart structure 360 are received within an anatomic simulator 300, as shown in FIG. 6B, the heart structure 360 may urge the lung model 350 into a configuration that may be more representative of a lung's actual configuration within a patient. Additionally, the presence of the heart structure 360 may prevent the lung model 350 from expanding into certain spaces within the volume defined in part by the constraint panels of the anatomic simulator 300, thereby more accurately reproducing the physical constraints on an actual lung as it expands. That is, the heart structure may be configured to constrain motion of the organ model when the organ model moves within the volume defined in part by the constraint panels. For example, without a heart structure, a lung model may expand radially outward, which may not be representative of standard respiration. In contrast, inclusion of a heart structure within an anatomic simulator may urge the lung model to expand downwards, which may be more realistic and more representative of standard respiration. In some embodiments, a heart structure (or other structure disposed within the volume of an anatomic simulator) may include radiopaque portions that may aid in registration in a manner similar to that discussed above with regards to radiopaque portions of the constraint panels.

In some embodiments, the one or more additional structures may include inserts or panels (not shown) for shifting a position of an organ model within the anatomic simulator. For example, in some embodiments where a shift in patient anatomy between pre-operative imaging of the anatomy and a patient procedure can be predicted, the inserts may in placed within the simulator after pre-operative images have been captured. Accordingly, the organ model can be shifted to simulate a predicted anatomical shift that would be experienced during a true patient procedure. In some embodiments, the inserts can be placed below, above, or on any side of the organ model shifting the organ as desired.

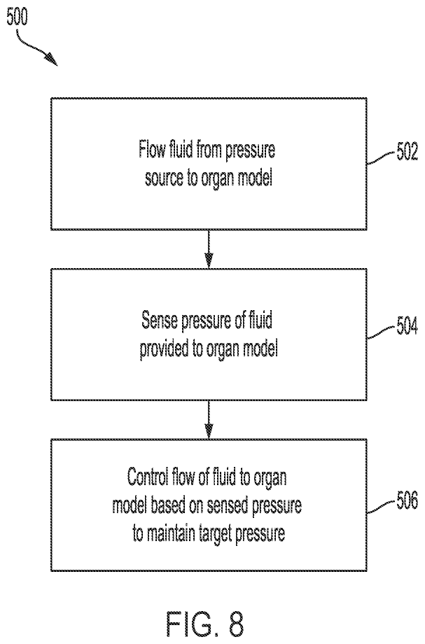

FIG. 7 is a schematic of one embodiment of an anatomic simulation assembly 400. The assembly 400 includes an anatomic simulator 402 configured to receive an organ model. A pressure source 404 is in fluid communication with the anatomic simulator 402 and is configured to flow a fluid to the organ model. A pressure source may include a volume of compressed fluid (e.g., a pressurized cylinder), a positive displacement pump, a bellows pump, or any other suitable component configured to provide pressurized fluid. The fluid in the anatomic simulation assembly may be a gas (e.g., air), a liquid (e.g., water), a gas/liquid mixture, or any other suitable fluid. Along the flow path between the pressure source 404 and the anatomic simulator 402 are pneumatic controls 406. Pneumatic controls 406 may include controllable valves (e.g., check valves, ball valves, butterfly valves, gate valves, needle valves, variable flow control valves, etc.), restrictors (e.g., variable flow resistance restrictions), regulators, or any other suitable pneumatic component configured to alter and/or control a pressure and/or flow rate of a fluid from the pressure source to the organ model. It should be appreciated that while pneumatic controls may be used when the fluid from the fluid source is a gas, other types of controls may be used with other fluids. For example, if the anatomic simulator is configured to receive a liquid, the assembly may include hydraulic controls instead of or in addition to the pneumatic controls shown in the figure.

The assembly 400 additionally includes a pressure sensor 408 configured to sense a pressure of the fluid provided to the organ model. Although FIG. 7 shows the pressure sensor 208 coupled to the anatomic simulator 402, in other embodiments a pressure sensor may be configured to sense a pressure at a different point along the flow path between the pressure source 404 and the anatomic simulator 402. In some embodiments, an anatomic simulation assembly may include multiple pressure sensors to enable pressure sensing at different points within the assembly. For example, pressure sensors may be configured to sense pressures associated with a pressure source, one or more pneumatic (or hydraulic) controls, an opening of the organ model, and/or any suitable portion of an anatomic simulator. Pressure sensors may also be configured to sense pressures associated with the flow path between any of the above-mentioned components of an anatomic simulation assembly. Additional sensors other than pressure sensors may also be included in an anatomic simulation assembly, including but not limited to flow rate sensors, temperature sensors, humidity sensors, chemical sensors, or any other suitable sensor.

The assembly 400 further includes a processor 410 operatively coupled to the pressure sensor 408 and at least one of the pneumatic controls 406. The processor may be operatively coupled with associated non-transitory computer readable memory that includes instructions that when executed cause the assembly to perform any of the methods described herein. The processor 410 may additionally be coupled to the pressure source 404 in some embodiments. The processor is configured to operate in different modes, which may be selected by a user through a user interface 412. A user interface 412 may be connected to a processor via a wired or wireless connection. A user interface may include a combination of buttons, dials, and/or switches; a touch screen; an application for a smart phone; or any other suitable interface configured to receive input from a user. An anatomic simulation assembly may be configured to operate in different operating modes, which may be selected based on ser input received through the user interface.

In some embodiments, a system may be configured to operate in a normal breathing mode. In such an operating mode the processor 410 controls the flow of fluid to cyclically increase and decrease the pressure within an organ model disposed within the anatomic simulator 402, thereby causing the organ model to expand and retract in a manner representative of steady respiration. Specifically, the processor 410 controls the flow of fluid from the pressure source 404 to the organ model using the one or more pneumatic controls 406 based at least partially on a sensed pressure from the pressure sensor 406. In some embodiments, fluid may be actively forced (e.g., pumped) into the organ model, and may be expelled passively by opening a valve and relying on the natural elasticity of the organ model and/or the restoring force of an elastic member of the anatomic simulator to force fluid out of the organ model. Alternatively, in some embodiments a flow resistance between the pressure source may be increased by closing a valve or increasing a flow resistance along a flow path extending from the pressure source to the organ model to reduce a flow of fluid to the organ model. Similar to the above, the natural elasticity of the organ model and/or the restoring force of an elastic member of the anatomic simulator may then force fluid out of the organ model. Of course, embodiments in which the pressure source and/or pneumatic controls are operated to actively force fluid into and out of the organ model are also contemplated.

In some embodiments, normal breath mode may be operated in open-loop control, in which the sensed pressure from the pressure sensor 406 may not be used as feedback. In some embodiments, the processor 410 may control the flow of fluid based on one or more parameters beyond the sensed pressure from the pressure sensor 406. These additional parameters may be default program settings, or may be editable by a user, such as through the user interface 412. The additional parameters may include a cycle rate parameter such as a number of breaths per minute, a pressure limit such as a maximum pressure, a pressure rate limit such as a maximum rate of pressure increase, a flow rate limit, or any other suitable parameter. In some embodiments, the controls of an anatomic simulation assembly may be representative of the controls of a ventilator, and thus may be able to replicate some or all of the functions of a ventilator.

In some embodiments, an assembly may also be selectively operated in a breath hold mode by a user. In such an embodiment, the processor 410 controls the flow of fluid to maintain a target pressure within the organ model. For example, the pressure sensor 408 may sense a pressure within the organ model and/or other portion of a fluid flow path connected to the organ model. The processor 410 may then control the flow of fluid to the organ model to maintain a target pressure based on the sensed pressure until a certain condition is determined (e.g., a time limit is reached, or additional user input is received through the user interface 412). In some embodiments, the processor may control the flow fluid to the organ model to maintain the target pressure if the sensed pressure is greater than or equal to a threshold pressure. It should be appreciated that depending on the embodiment the target pressure may be the same as or greater than the threshold pressure in various embodiments. The target pressure and/or the threshold pressure may be received through the user interface 412. The sensed pressure from the pressure sensor 408 (and/or other information from any other sensors) is used in a closed-loop feedback control architecture to maintain the target pressure within the organ model once the threshold pressure is exceeded. In breath hold mode, the processor 410 controls the pressure source 404 and/or the pneumatic controls 406 to make adjustments to maintain the target pressure, potentially accommodating for the presence of any defects in the organ model. For example, if the organ model includes small perforations such that stopping all flow into the organ model would result in a continual pressure decrease due to a small leak, the processor may instead control the pressure source and/or the pneumatic controls to maintain a corresponding fluid flow into the organ model to compensate for the leaks, thereby maintaining the pressure within the organ model at the target pressure. In this way, closed-loop feedback control (whether implemented in the breath hold mode, or in a different mode) may enable robust performance, even with an imperfect organ model.

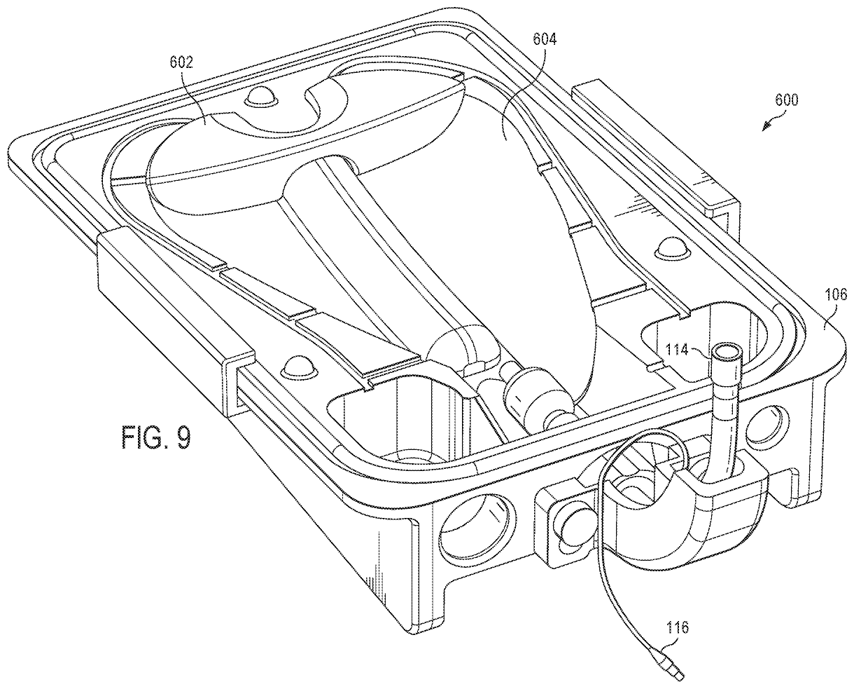

FIG. 8 is a flowchart of one embodiment of a method 500 of simulating anatomic motion. As described above, an anatomic simulation assembly may include a breath hold mode, in which motion of a lung model is reduced to enable delicate tasks. To enable a breath hold, fluid is first provided from a pressure source to an organ model within an anatomic simulator, such as at 502. As described above, fluid may be provided to the organ model using any suitable pressure source and/or any suitable pneumatic controls, as the present disclosure is not limited in this regard. At 504, the pressure of the fluid provided to the organ model is sensed, for example using one or more pressure sensors. The pressure may be sensed at the organ model, at the pressure source, or at any point along the flow path connecting the pressure source and the organ model. At 506, the flow of fluid to the organ model is controlled based at least partially on the sensed pressure to maintain a target pressure within the organ model. The pressure may be sensed and controlled continuously to ensure that the organ model maintains the target pressure as long as the breath hold mode is enabled. In this way, imperfections within the organ model (e.g., small perforations that lead to leaks) may be accommodated for.

While only two control modes are described above (i.e., cyclic normal breathing and breath hold), it should be understood that a user may selectively switch operation of an anatomic simulation assembly between any number of desired operating modes as the disclosure is not so limited.

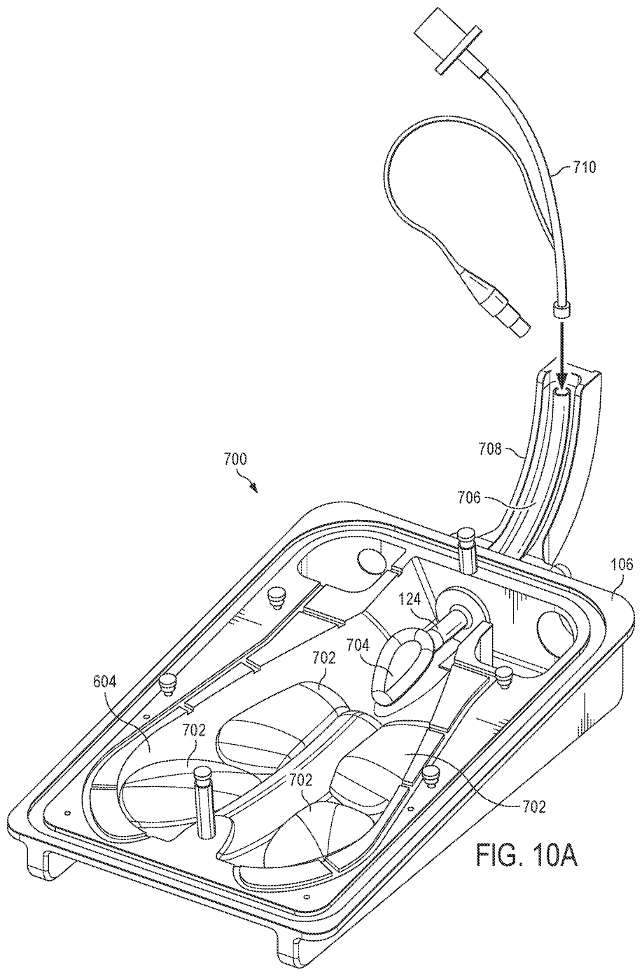

FIG. 9 is a perspective view of an example of an anatomic simulator 600 including a simulation insert member 602. In this example, anatomic simulator 600 includes the base 106 which has a recessed tray 604 on which at least a portion of an organ model (e.g. organ model 250 of a set of lung) or a real organ (e.g. a set of real lungs) may be positioned. The simulation insert member 602 may be positioned on the recessed tray 604 to simulate various anatomic conditions or deformations. Simulation inserts may be used, for example, to simulate a pre-operative image to real body divergence. For example, to simulate procedure in which a pre-operative CT image diverges from the intraoperative patient anatomy, a simulation insert may be inserted, with the organ model, into the recessed tray of the base to simulate a divergence between a pre-operative image of the organ model and the in-procedure configuration of the organ model. In this example, the simulation insert member 602 positioned in the inferior region of the tray 604 may constrain expansion of the lung model in an inferior direction.

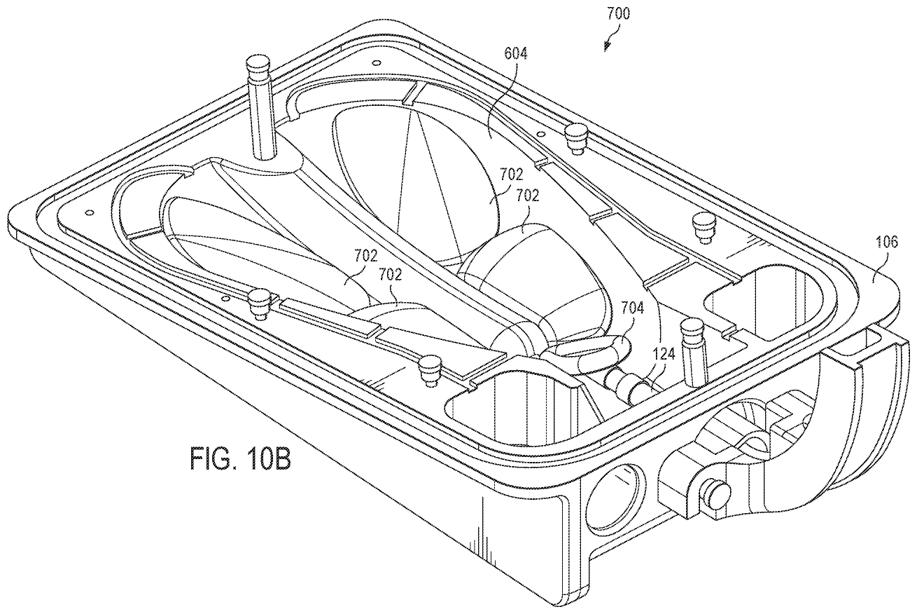

FIGS. 10A and 10B are perspective views of an example of an anatomic simulator 700 including simulation insert members 702 in the recessed tray 604 and a laryngeal mask airway (LMA) device 704 coupled to the fluid outlet 124. The simulation insert members 702 may be positioned on the recessed tray 604 to simulate various anatomic conditions or deformations as described above. The LMA device 704 may be used for the exchange of fluid (e.g., air) to and from an organ model (e.g. organ model 250 of a set of lung) or a real organ (e.g. a set of real lungs) that may be positioned in the base 106. The LMA device 704 may, for example, be seated above the trachea of the organ model such that fluid passes from the LMA device 704 into the organ model. In this example, the fluid outlet 124 may be coupled between the LMA device 704 and tubing 706. The tubing 706 may be constrained by a fixture 708 that maintains the end of the tubing in a predetermined configuration for attachment to an endotracheal (ET) tube 710 or other fluid source.

Alternatively, the tubing 706 may be eliminated and the LMA device 704 and fluid outlet 124 may be directly coupled to the ET tube 710. In this alternative, the ET tube 710 may be laterally constrained by the fixture 708. Further, the ET tube 710 may be constrained in both axial (e.g., in/out motion) and radial (e.g., lateral left/right motion) degrees of freedom by one or more flanges, rigid concentric structures, an/or the surrounding concentric base wall at an entry port to the base 106. In this example, the LMA device 704 may remain substantially fixed in orientation and position. In some examples, the ET tube 710 may be unconstrained in axial motion (e.g., in/out motion), allowing for translational positioning of the LMA device 704 during a simulated procedure. The LMA device 704 either may be suspended in the trachea of the organ model, with a position and orientation influenced by the base 106 and other structures or may be configured to be compliant so as to allow the positioning to be prescribed by the anatomy of the trachea of the organ model.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computing device or distributed among multiple computing devices. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Also, the disclosed systems, and associated one or more processors, may have one or more input and output devices. These devices can be used, among other things, to present a user interface. Examples of output devices that can be used to provide a user interface include display screens for visual presentation of output and speakers or other sound generating devices for audible presentation of output. Examples of input devices that can be used for a user interface include keyboards, individual buttons, and pointing devices, such as mice, touch pads, and digitizing tablets. As another example, a computing device may receive input information through speech recognition or in other audible format.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the embodiments described herein may be embodied as a computer readable storage medium (or multiple computer readable media) (e.g., a computer memory, one or more floppy discs, compact discs (CD), optical discs, digital video disks (DVD), magnetic tapes, flash memories, RAM, ROM, EEPROM, circuit configurations in Field Programmable Gate Arrays or other semiconductor devices, or other tangible computer storage medium) encoded with one or more programs that, when executed on one or more computers or other processors, perform methods that implement the various embodiments discussed above. As is apparent from the foregoing examples, a computer readable storage medium, which may also be referred to as a computer readable memory, may retain information for a sufficient time to provide computer-executable instructions in a non-transitory form. Such a computer readable storage medium or memory can be transportable, such that the program or programs stored thereon can be loaded onto one or more different computing devices or other processors to implement various aspects of the present disclosure as discussed above. As used herein, the term “computer-readable storage medium”, or similar term, encompasses only a non-transitory computer-readable medium that can be considered to be a manufacture (i.e., article of manufacture) or a machine. Alternatively or additionally, the disclosure may be embodied as a computer readable medium other than a computer-readable storage medium, such as a propagating signal.

The terms “program” or “software” are used herein in a generic sense to refer to any type of computer code or set of computer-executable instructions that can be employed to program a computing device or other processor to implement various aspects of the present disclosure as discussed above. Additionally, it should be appreciated that according to one aspect of this embodiment, one or more computer programs that when executed perform methods of the present disclosure need not reside on a single computing device or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

1. An anatomic simulator comprising:
a first constraint panel configured to rotate about a first axis;
a second constraint panel configured to rotate about a second axis, wherein the first and second constraint panels at least partially define a variably sized volume configured to receive an organ model; and
at least one elastic member operatively coupling the first and second constraint panels, wherein the at least one elastic member is configured to provide a restorative force when the first and second constraint panels are rotated to increase a size of the volume.

2. The anatomic simulator of claim 1, wherein the organ model expands or collapses within the variably sized volume.

3. The anatomic simulator of claim 1, wherein when the organ model is pressurized, the first constraint panel rotates about the first axis in a first direction and the second constraint panel rotates about the second axis in a second direction.

4. The anatomic simulator of claim 3, wherein when the organ model is depressurized, the first constraint panel rotates about the first axis in a third direction and the second constraint panel rotates about the second axis in a fourth direction, wherein the third direction is opposite the first direction, and wherein the fourth direction is opposite the second direction.

5. The anatomic simulator of claim 1, wherein the restorative force provided by the at least one elastic member biases the first constraint panel and the second constraint panel towards a retracted configuration.

6. The anatomic simulator of claim 1, further comprising the organ model disposed within the variably sized volume.

7. The anatomic simulator of claim 1, wherein the organ model is a lung model.

8. The anatomic simulator of claim 1, further comprising a structure disposed within the variably sized volume.

9. The anatomic simulator of claim 8, wherein the structure is a heart structure configured to constrain motion of the organ model when the organ model moves within the variably sized volume.

10. The anatomic simulator of claim 9, wherein at least a portion of the heart structure is radiopaque.

11. The anatomic simulator of claim 8, wherein the structure is an insert configured to shift the organ model within the variably sized volume.

12. The anatomic simulator of claim 1, further comprising a base configured to support the organ model when the organ model is received within the anatomic simulator, wherein the first constraint panel is rotatably coupled to the base, and wherein the second constraint panel is rotatably coupled to the base.

13. The anatomic simulator of claim 1, wherein at least a first portion of the first constraint panel is radiopaque, and wherein at least a second portion of the second constraint panel is radiopaque.

14. The anatomic simulator of claim 13, wherein the first and second portions are representative of anatomic features.

15. The anatomic simulator of claim 1, further comprising a pressure source configured to flow a fluid to the organ model when the organ model is received within the variably sized volume.

16. The anatomic simulator of claim 15, further comprising:
one or more pneumatic controls disposed along a flow path between the pressure source and the organ model when the organ model is received within the variably sized volume; and
a processor operatively coupled to the one or more pneumatic controls, wherein the processor is configured to control the flow of fluid from the pressure source to the organ model with the one or more pneumatic controls.

17-64. (canceled)

65. An anatomic simulation assembly, the assembly comprising:
an anatomic simulator configured to receive an organ model;
a pressure source in fluid communication with the anatomic simulator, wherein the pressure source is configured to flow a fluid from the pressure source to the organ model when the organ model is received within the anatomic simulator; and
a laryngeal mask airway device received within the anatomic simulator and configured to engage the organ model, wherein the fluid from the pressure source flows through the laryngeal mask airway device and into the organ model.

66. The anatomic simulation assembly of claim 65 wherein the laryngeal mask airway device is constrained from relative motion relative to the anatomic simulator.

67. The anatomic simulation assembly of claim 66 wherein the laryngeal mask airway device is constrained from axial motion relative to the anatomic simulator.

68. The anatomic simulation assembly of claim 65 further comprising a simulation insert member positioned within an interior volume of the anatomic simulator to provide a constraint on the organ model.
